(19) United States
(12) Patent Application Publication
(10) Pub. No.: US 2013/0178855 A1
(43) Pub. Date: Jul. 11, 2013

(54) IMPLANTABLE HEARING AID AND SYSTEM AND METHOD FOR IMPLANTING THE SAME

(71) Applicants: Gérard Loquet, Lausanne (CH); Achim Kitschmann, Basel (CH); Stefan Menzl, Jona (CH)

(72) Inventors: Gérard Loquet, Lausanne (CH); Achim Kitschmann, Basel (CH); Stefan Menzl, Jona (CH)

(73) Assignee: ADVANCED BIONICS AG, Zug (CH)

(21) Appl. No.: 13/698,789

(22) PCT Filed: May 17, 2010
(86) PCT No.: PCT/EP10/56749

§ 371 Date: Mar. 7, 2013

(51) Int. Cl. A61B 17/16; A61B 19/00; A61N 1/36

(52) U.S. Cl. CPC A61B 17/1679; A61N 1/36032; A61B 19/2203
USPC 606/80; 607/57; 381/326

(57) ABSTRACT

An at least partially implantable hearing aid, having: a microphone arrangement (74) for capturing audio signals from ambient sound; an audio signal processing unit (66) for processing the audio signals captured by the microphone arrangement; an implantable actuator unit (54) for stimulating the patient's hearing according to the processed audio signals; a tube (24) for being implanted into the patient's temporal bone (26), the tube having an engagement structure (46) at the outer surface for engagement into a fixation structure (44) milled into the patient's temporal bone, wherein the tube comprises means (36) for receiving the actuator unit in an implanted condition of the tube from the proximal end of the tube and means for fixing the actuator unit in a final position relative to the tube, in which final position the actuator unit extends beyond the distal end of the tube.

### BACKGROUND OF THE INVENTION

1. Field of the Invention

The present invention relates to an at least partially implantable hearing aid and to a system and method for implanting an actuator of such hearing aid.

2. Description of Related Art

Fully or partially implantable hearing aids, such as cochlear implants and middle-ear hearing implants, require surgery for implantation. The anatomy of both the middle ear and the inner ear is difficult to approach, and most of the implantable hearing aids and cochlear implants require a large mastoidectomy and a tympanotomy, and in addition, often create more or less severe damage to the natural path of the hearing function (cochlear structures, ossicular chain, etc.). Besides, the available space is always a challenge and hence most of the products on the market include a compromise between design and functionality.

An example of an implantable hearing aid comprising an electro-mechanical actuator is described in International Patent Application Publication WO 2006/058368 A1. An example of a fixation element for fixing the electro-mechanical actuator of such hearing aid within the patient's skull is described in International Patent Application Publication WO 2006/058367 A1.

U.S. Patent Application Publication 2006/0247517 A1 relates to a method for implanting a cochlear implant, wherein the position of a surgical instrument used for opening an access to the cochlea of the patient is monitored by an optical position sensor, with the surgical instrument being provided with a corresponding optical emitter. In addition, also the position of the patient's skull is monitored. with a fiducial frame comprising optical trackers/emitters being fixed at the patient's skull. Thereby, the position of the surgical instrument relative to the patient's skull can be monitored during surgery. By using an image of the patient's skull structure taken prior to surgery, the position of the surgical instrument can be monitored by the surgeon during surgery.

U.S. Pat. No. 7,366,562 B1 relates to a surgical navigation system wherein a surgical instrument is fixed at a non-invasive dynamic reference frame fixed at the patient's body for use in implanting a pacemaker.

German Patent Application DE 199 02 273 A1 relates to a system for implanting dental implants, wherein a computer-based navigation system is used for displaying the position of the drilling device in three-dimensions in X-ray images of the jaw bones, with a dynamic reference frame attached to the patient's teeth being used.

Current solutions used for implanting hearing aids are very invasive and can only be performed under general anaesthesia, which gives no chance to test, and if necessary, to adjust the implanted parts of the hearing aid before closing the incision. Current implantable hearing aids need a pretty complex, difficult to execute and long duration surgery for implantation; moreover, the requirements on surgical skills of the operator are very high.

### SUMMARY OF THE INVENTION

It is an object of the invention to provide for an implantable hearing aid which is suitable for being implanted in a particularly easy and fast manner and which nevertheless provides for reliable performance. It is also an object of the invention to provide for a corresponding method and system for implanting such hearing aid.

According to the invention, these objects are achieved by a hearing aid and a method as described herein.

The invention is beneficial in that, providing the hearing aid with a tube for being implanted into the patient's temporal bone, which comprises a structure at the outer surface for engagement with a fixation structure drilled into the patient's temporal bone and which comprises means for receiving the actuator of the hearing aid in an implanted condition of the tube from the proximal end and means for fixing the actuator in a final position relative to the tube, in which final position the actuator extends beyond the distal end of the tube, a compact design of the implanted part of the hearing aid is enabled, and the hearing aid, in particular, the actuator thereof, can be implanted in a simple, reliable and fast manner. The hearing aid of the invention enables implantation by using an automated drilling device, whereby precision of the implant position can be enhanced, duration of the implantation can be reduced (for example, to less than one hour), thereby reducing the risk for the patient, and the requirements regarding the skills of the operator can be reduced. Implantation may even be carried-out under local anaesthesia. Compared to current solutions, the mechanical set-up of the hearing aid of the invention is simpler, more robust and better protected against impact (for example, as caused by a strike against the patient's head resulting from a golf ball or baseball, etc.).

These and further objects, features and advantages of the present invention will become apparent from the following description when taken in connection with the accompanying drawings which, for purposes of illustration only, show several embodiments in accordance with the present invention.

### BRIEF DESCRIPTION OF THE DRAWINGS

FIG. 1 is a schematic representation of an implantation system according to the invention;

FIG. 2 is a schematic representation of how the system of FIG. 1 is used for microsurgery during implantation;

FIG. 3 is a cross-sectional view of a patient's ear region, with the tube of a hearing aid having been implanted;

FIG. 4 is an enlarged view of the distal end of the tube of FIG. 3 with the actuator having been inserted into the tube of the hearing aid;

FIG. 5 is a schematic view of how the tube of the hearing aid is used during implantation; and

FIG. 6 is a longitudinal sectional view of the interior of the tube of the hearing aid, when receiving an electronic component of the hearing aid.

### DETAILED DESCRIPTION OF THE INVENTION

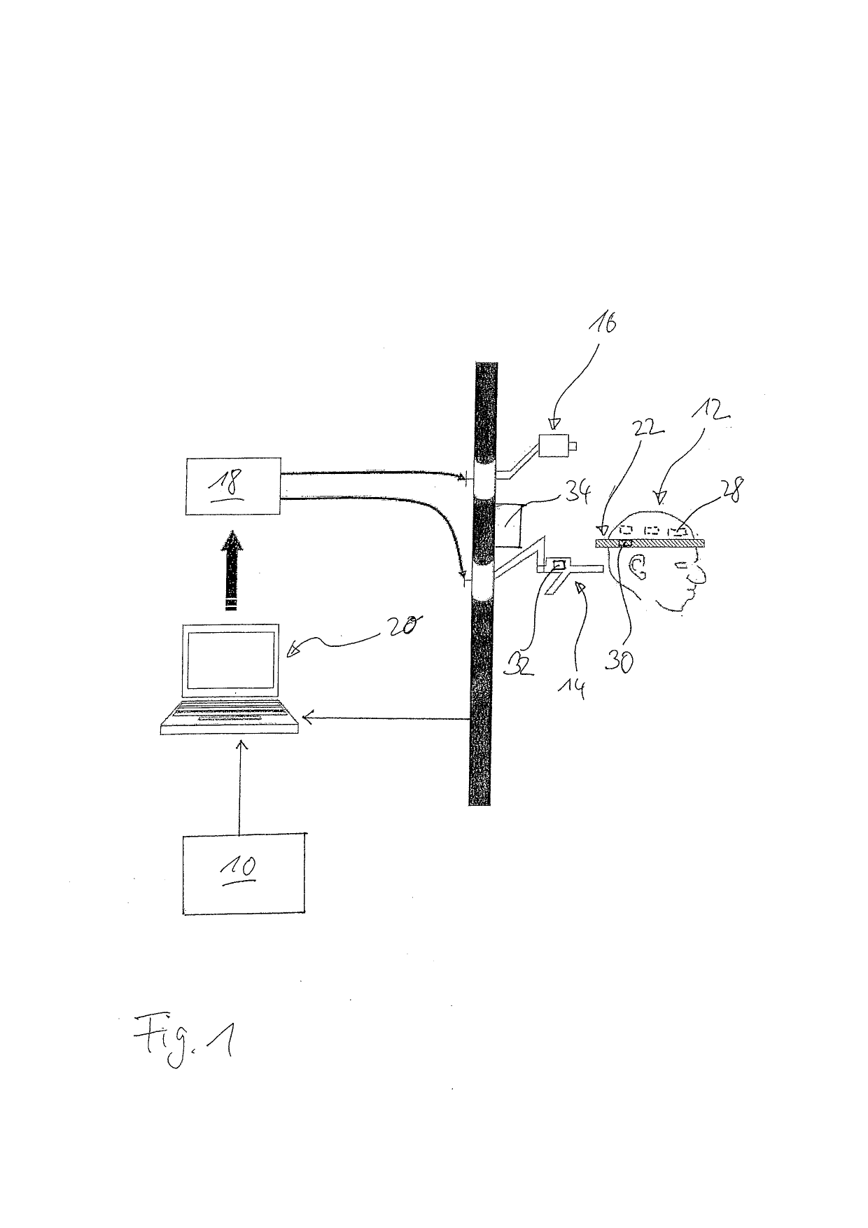

FIG. 1 is a schematic view of a system for implanting a fully-implantable hearing aid or the implantable part of a partially implantable hearing aid; in the latter case, the implantable part comprises at least the actuator unit of the hearing aid. The system comprises a device 10 for taking an image of the structure of the skull 12 of a patient, which is usually a Computed Tomography (CT) or Magnetic Resonance Imaging (MRI) device, a mechanical drilling tool 14, a laser drilling tool 16, a robotic driver 18 for driving the drilling tools 14, 16, a controller 20 for controlling the robotic driver 18, and a device 22 for fixing the patient's skull 12 relative to the drilling tools 14, 16. The controller 20 usually is a computer. The object to be implanted by the implanting system is a tube for holding the actuator of the hearing aid.

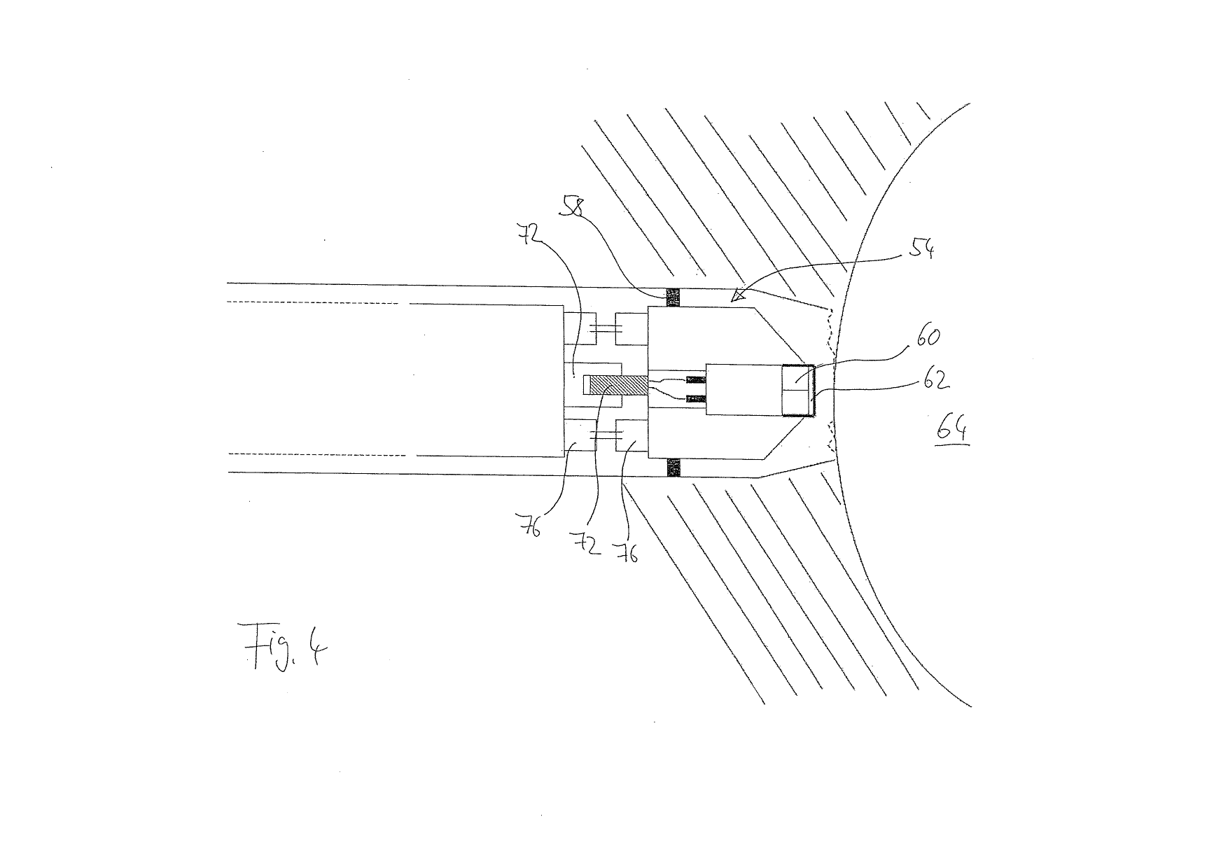

An example of such a tube 24 is shown in FIG. 3 in its implanted state. The tube 24 is implanted into the patient's temporal bone 26 at a predefined target position. The target position is defined by the surgeon according to the image of the patient's skull structure taken by the device 10 and up-loaded onto the controller 20. Preferably, the image of the patient's skull structure is three-dimensional. Prior to taking the image of the patient's skull structure, a plurality of markers (which are schematically shown at 28 in FIG. 1) are attached to the patient's skull 12 in order to define reference positions on the image.

The system is capable of monitoring the position of the drilling tools 14, 16, relative to the patient's skull structure. To this end, the system comprises a tracker system comprising at least one emitter 30 to be placed at the mechanical drilling tool 14 and at least one emitter 32 to be placed at the patient's skull 12 and a position sensor 34 for receiving the signals of the emitters 30, 32. The at least one emitter 30 typically forms part of a head frame which also serves as the device 22 for fixing the patient's skull relative to the drilling tool 14. The signal of the position sensor 34 is supplied to the controller 20 so as to precisely reach the predefined target position of the tube 24 relative to the patient's skull structure by controlling the robotic driver 18 according to the image of the patient's skull structure and according to the monitored position of the mechanical drilling tool 14.

Preferably, the tracker system is optical.

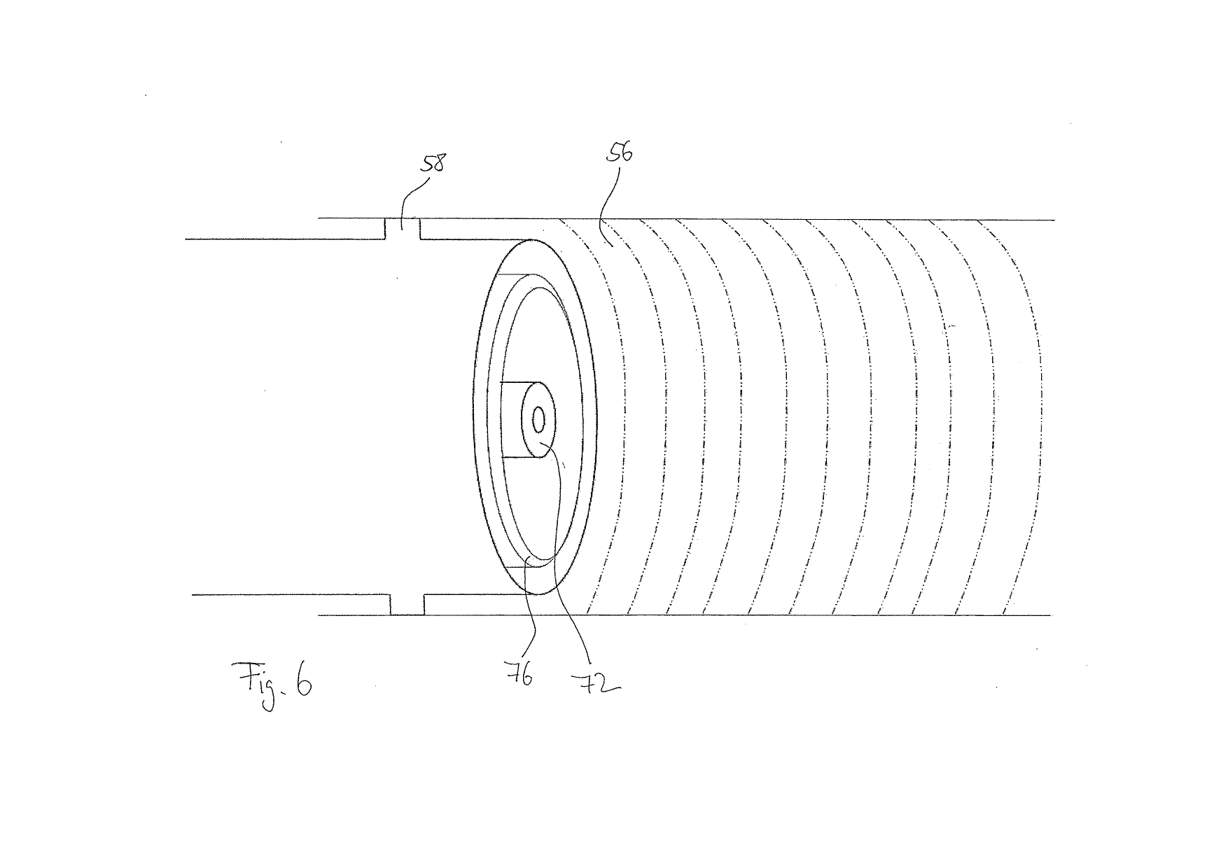

FIG. 5 is a schematic view of how the tube 24 of the hearing aid is used during implantation together with the mechanical drilling tool 14. The distal section of the mechanical drilling tool 14 comprises a drilling shaft 36 carrying a drilling head 38 which preferably is made of titanium, wherein the drilling head 38 extends beyond the distal end of the tube 24 and wherein the shaft 36 is rotated relative to the tube 24 via a coupling piece 40 which forms part of or is connected to a rotating member 42. Thus, during implantation, the drilling shaft 36 extends through the interior of the tube 24 which is thereby gradually inserted into a hole in the patient's temporal bone created by the drilling head 38. Preferably, the outer surface of the drilling shaft 36 is designed as a no-end screw that will cause bone residues to move outside the cavity during drilling. The drilling shaft 36 and the drilling head 38 are hollow so that, during the drilling action, some compact bone core will be left, whereas the milled material is moved outside by the no-end thread at the outer surface of the drilling shaft 36 (the outer surface of the drilling shaft 36 is close to the inner wall of the tube 24).

The movement of the mechanical drilling tool 14 is controlled by the controller 20 in such a manner that the tube 24 is gradually brought into the predefined target position within the temporal bone 26 based on the image of the skull structure and the monitored actual position of the drilling tool 14 relative to the skull 12.

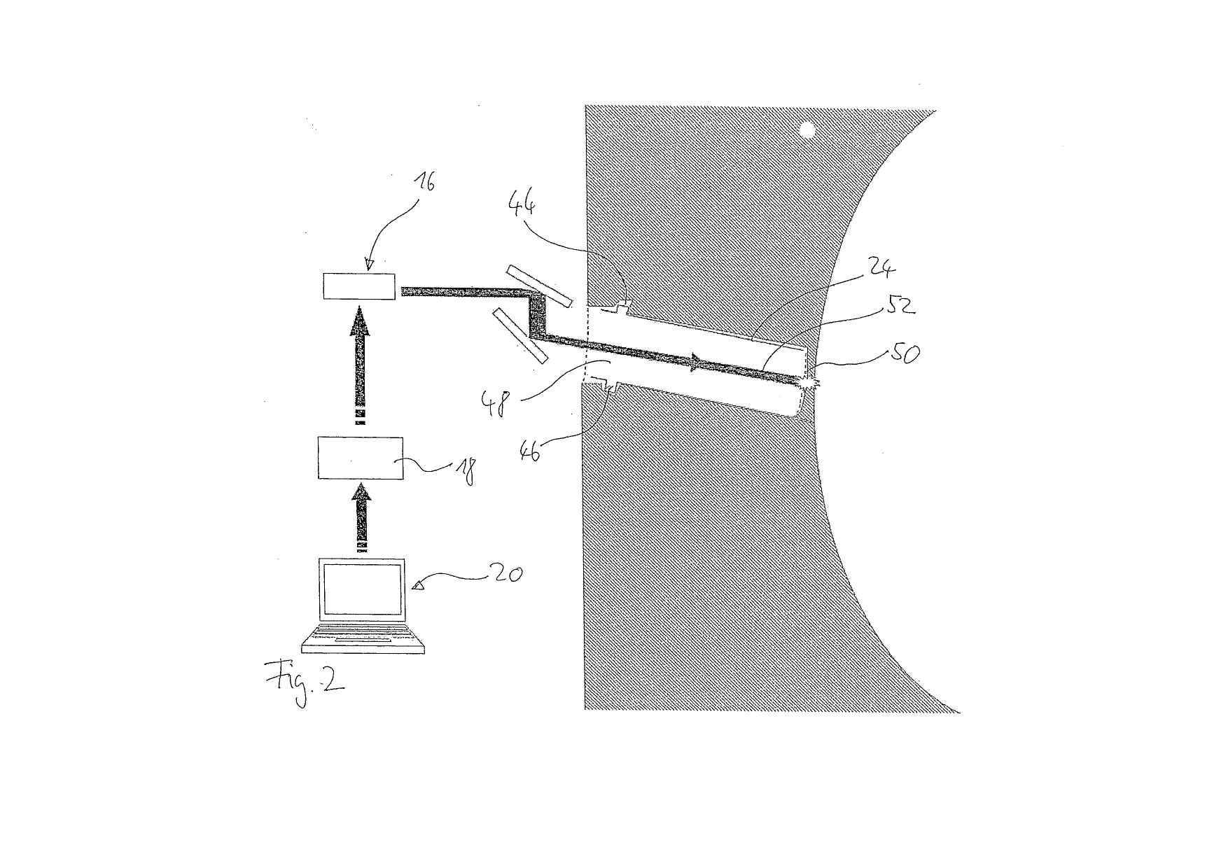

Preferably, the drilling action is performed in such a manner that the hole in the temporal bone 26 is provided with a fixation structure, such as some kind of thread 44, into which a mating structure of the outer surface of the tube 24, such as projections 46, engage, for example, in a bayonet-type manner, see FIG. 2. The projections 46 may serve not only to prevent axial movement of the tube 24, but also rotational movement of the tube 24. Thereby the tube 24 is fixed in the final position which corresponds to the predefined target position. The outer surface of the tube 24 may be coated with a layer of calcium phosphate or hydroxyapatite, which materials promote fast growing-together of the bone structure and the tube 24.

Once the tube 24 has reached its final position, the drilling head 38 and the drilling shaft 36 are withdrawn from the tube 24.

Thereafter, the laser drilling tool 16 is used, as shown in FIG. 2, to finalize the drilling action at the distal end of the cavity 48 created by the mechanical drilling tool 14. Thereby, residual cochlear bone 50 may removed by a laser beam 52 which is emitted by the laser drilling tool 16 and which passes through the interior of the tube 24. Preferably, the laser drilling tool 14 comprises a femto-laser. The laser beam 52 is controlled by the laser drilling tool 16 in such a manner that a coupling site for an actuator unit 54 is created at the patient's middle ear or inner ear.

Once the laser drilling action is terminated, the tube 24 is filled with the functional components of the hearing aid. The first component to be introduced into the tube is the actuator unit 54. The inner surface of the tube 24 comprises a fixation structure for engaging with a mating structure of the outer surface of the actuator unit 54 for locking actuator unit 54 with the tube 24. As indicated in FIG. 6, the fixation structure of the tube 24 may be a helicoidal groove 56 for engaging in a bayonet-type manner with projections 58 provided at the outer surface of the actuator unit 54 as the mating structure.

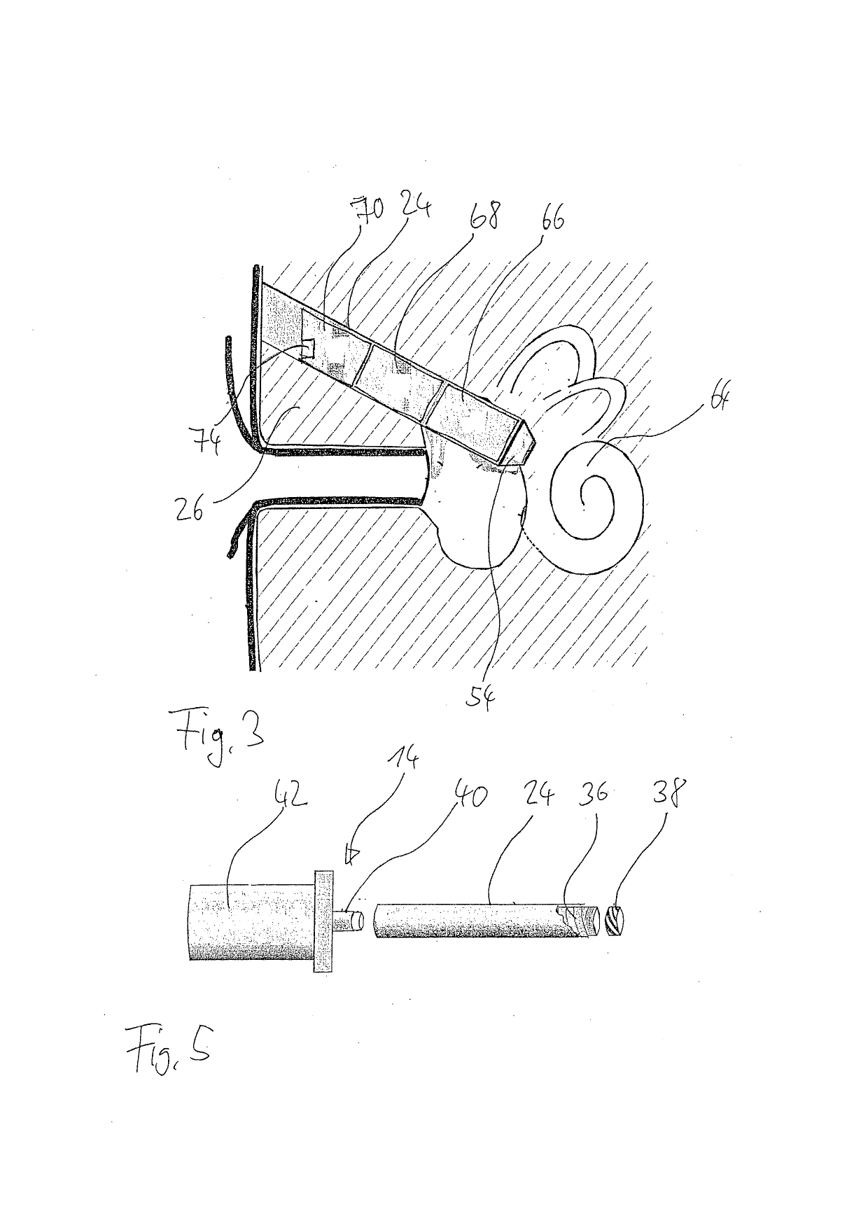

According to the embodiment shown in FIG. 4, the actuator unit 54 comprises a transducer 60 with a membrane 62 which acts directly on the perilymph of the cochlea 64. According to an alternative embodiment, the actuator unit 54 may comprise a cochlear electrode.

FIG. 3 shows an example of a fully implantable hearing aid. In this case, the tube 24 is subsequently filled with a signal processing unit 66, a power supply unit 68 and a microphone unit 70, wherein the actuator unit 54, the audio signal processing unit 66, the power supply unit 68 and the microphone unit 70 are arranged in a lined-up configuration within the tube 24 and wherein each of the units 54, 66, 68, 70 is provided at each side which is adjacent to one of the other units with an electrical connector 72 which engages with its mating counterpart at the adjacent unit. Each of the units 54, 66, 68, 70 is provided with a mating structure, such as projections 58, at its outer surface for engagement with the fixation structure 56 of the tube 24 for locking the respective unit within the tube 24.

The microphone 70 comprises a microphone arrangement 74 for capturing audio signals from ambient sound, the audio signal processing unit 66 is for processing the audio signals captured by the microphone arrangements 72, and the actuator unit 54 is for stimulating the patient's hearing according to the processed audio signals. The power supply unit 68 is for supplying the units 54, 66 and 70 with power.

In the embodiment shown in FIG. 4 each of the electrical connectors 72 is aligned with regard to the center of the tube 24 and is coaxially surrounded by an anchor element 76 which engages with a mating anchor element 76 of the adjacent unit in a manner so as to align the electrical connector 72, see also FIG. 6.

While a circular cross-section of the tube 24, as shown in the figures, is preferred, non-circular cross-sections are also conceivable.

If the hearing aid is designed as partially implantable device, at least one of the units 66, 68 and 70 may be located outside the patient's body, as it is known in the art, and then it would not be located within the tube 24.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

1. An at least partially implantable hearing aid, comprising:
a microphone arrangement for capturing audio signals from ambient sound;
an audio signal processing unit for processing the audio signals captured by the microphone arrangement;
an implantable actuator unit for stimulating a patient's hearing according to the processed audio signals;
a tube for being implanted into a patient's temporal bone, the tube comprising a engagement structure at an outer surface for engagement into a fixation structure milled into the patient's temporal bone;
wherein the tube comprises means for receiving the actuator unit in an implanted condition of the tube from a proximal end of the tube and means for fixing the actuator unit in a final position relative to the tube, in which final position the actuator unit extends beyond a distal end of the tube.

2. The hearing aid of claim 1, wherein the tube has a circular cross section.

3. The hearing aid of claim 1, wherein the outer surface of the tube is coated with calcium phosphate or hydroxyapatite to promote fast growing-together of bone structure with the tube.

4. The hearing aid of claim 1, wherein the engagement structure comprises projections for engagement with a thread or a groove allowing for a bayonet engagement of the projections.

5. The hearing aid of claim 1, wherein the actuator unit comprises a membrane for directly acting on the perilymph of the cochlea.

6. The hearing aid of claim 1, wherein the actuator unit comprises a cochlear electrode.

7. The hearing aid of claim 1, wherein an inner surface of the tube comprises a fixation structure for engaging with a mating structure of an outer surface of the actuator unit for locking the actuator unit within the tube.

8. The hearing aid of claim 7, wherein the fixation structure of the tube is a helicoidal groove for engaging in a bayonet manner with projections provided at the outer surface of the actuator unit as the mating structure.

9. The hearing aid of claim 7, wherein the microphone arrangement is provided as part of a microphone unit which has a mating structure at an outer surface for engagement with the fixation structure of the tube for locking the microphone unit within the tube.

10. The hearing aid of claim 7, wherein the audio signal processing unit has a mating structure at an outer surface for engagement with the fixation structure of the tube for locking the audio signal processing unit within the tube.

11. The hearing aid claim 7, wherein a power supply unit is provided which has a mating structure at an outer surface for engagement with the fixation structure of the tube for locking the power unit within the tube.

12. The hearing aid of claim 11, wherein the actuator unit, the audio signal processing unit, the power supply unit and the microphone arrangement are arranged in a lined-up configuration within the tube and wherein each of the units is provided at each side which is adjacent to one of the other units with an electrical connector.

13. The hearing aid of claim 12, wherein each of the electrical connectors is aligned with regard to the center of the tube and is coaxially surrounded by an anchor element which engages with a mating anchor element of the adjacent one of the units in a manner so as to align the electrical connector.

14. A system for implanting an actuator unit of an at least partially implantable hearing aid, comprising:
means for taking an image of a patient's skull structure;
means for defining a target position of a tube within a patient's skull, said tube being provided for holding the actuator unit;
an automated drilling device for implanting the tube into the patient's temporal bone at the target position by using the image of the patient's skull structure;
means for fixing the patient's head relative to the automated drilling device;
wherein the automated drilling device comprises a rotating mechanical drilling tool having a distal section, said mechanical drilling tool being insertable into the tube in such a manner that the distal extends through the tube beyond the distal end of the tube for creating a cavity in the patient's temporal bone for receiving the tube, and wherein the automated drilling device comprises means for monitoring the position of the mechanical drilling tool relative to the patient's skull.

15. The system of claim 14, wherein the automated drilling device comprises a robotic driver for driving the mechanical drilling tool and a controller for controlling the robotic driver according to the image of the patient's skull structure and according to the monitored position of the drilling tool.

16. The system of claim 14, wherein the image of the patient's skull structure is three-dimensional.

17. The system of claim 16, wherein the means for taking an image of a patient's skull structure is a CT- or MRI-device.

18. The system of claim 14, wherein the system comprises a plurality of markers to be attached to the patient's skull when taking the image of a patient's skull structure.

19. The system of claim 14, wherein the means for monitoring the position of the mechanical drilling tool relative to the patient's skull structure comprises a tracker system comprising at least one emitter to be placed at the mechanical drilling tool and at least one emitter to be placed at the patient's skull and a position sensor for receiving the signal of the emitters.

20. The system of claim 19, wherein the tracker system is optical.

21. The system of claim 14, wherein the distal section of the mechanical drilling tool comprises a drilling shaft carrying a drilling head, wherein the drilling head extends beyond the front end of the tube, and wherein the shaft is rotated relative to the tube via a coupling piece.

22. The system of claim 21, wherein the outer surface of the drilling shaft is a no-end screw allowing bone residues to move outside the cavity during drilling.

23. The system of claim 21, wherein the drilling shaft and the drilling head are hollow.

24. The system of claim 14, wherein the automated drilling device comprises a laser drilling tool for emitting a laser drilling beam through an interior of the tube for creating a coupling site for the actuator unit at a patient's middle ear or inner ear.

25. The system of claim 24, wherein the laser drilling tool comprises a femto-laser.

26. The system of claim 14, wherein the means for fixing the patient's skull relative to the automated drilling device comprises a head frame.

27. A method for implanting an actuator unit of an at least partially implantable hearing aid, comprising the steps of:
taking an image of a patient's skull structure;
defining a target position of a tube within a patient's skull, said tube being provided for holding the actuator unit;
fixing the patient's skull relative to a drilling device;
implanting, by means of the drilling device and by using the image of the patient's skull structure, the tube into the patient's temporal bone, while monitoring the position of the tube during implantation relative to the patient's skull, and fixing the tube in the target position;
inserting at least the actuator unit into said tube and locking the actuator unit relative to the tube, with the actuator unit extending beyond a distal end of the tube.

28. The method of claim 27, wherein the drilling device is an automated robotic system which comprises a mechanical drilling tool controlled automatically according to the monitored position of the mechanical drilling tool relative to the patient's skull and the target position of the tube in the image of the patient's skull structure.

29. The method of claim 28, wherein the mechanical drilling tool comprises a drilling shaft carrying a drilling head, wherein, for implanting the tube, the tube is provided with the drilling shaft in such a manner that the drilling head extends beyond a front end of the tube, and wherein the shaft is rotated by the drilling device via a coupling piece.

30. The method of claim 29, wherein the drilling shaft is withdrawn from the tube prior inserting at least the actuator unit into the tube.

31. The method of claim 27, wherein, prior to inserting at least the actuator unit into the tube, residual cochlear bone is removed by a laser beam which is emitted by the drilling device and which passes through the tube.

32. The method of claim 27, wherein the tube is fixed in the final position by milling a fixation structure into the temporal bone and engaging an engagement structure of the outer surface of the tube into said fixation structure.

33. The method of claim 27, wherein the fixation structure is a thread.

34. The method of claim 27, wherein the fixation structure is a groove allowing for a bayonet engagement with projections provided at the outer surface of the tube as the engagement structure.

35. The method of claim 27, wherein the tube, in addition to the actuator unit, is filled also with a signal processing unit, a power supply unit and a microphone unit.
